(19) United States
(12) Patent Application Publication
(10) Pub. No.: US 2005/0205099 A1
(43) Pub. Date: Sep. 22, 2005

(54) Draping product having an adhesive edge for surgical interventions

(76) Inventors: Fredrik Gellerstedt, Onsala (SE); Helena Johansson, Goteborg (SE)

Correspondence Address: YOUNG & THOMPSON, 745 SOUTH 23RD STREET, 2ND FLOOR, ARLINGTON, VA, 22202, US

(21) Appl. No.: 10/508,553

(22) PCT Filed: Mar. 17, 2003
(86) PCT No.: PCT/SE03/00446

§ 371 Date: May 25, 2005

(30) Foreign Application Priority Data
Mar. 22, 2002 (SE) 0200886-0

(51) Int. Cl. A61F 5/37; A61B 19/00

(52) U.S. Cl. 128/849

(57) ABSTRACT

A draping product (1-4) for surgical interventions, which on its underside is coated with adhesive along at least one edge thereof (5, 6, 7, and 8 respectively), the coating (12) extending fully or partly along the edge. The adherence force against skin of the adhesive is greater than 0.5 N/25 mm and the adherence force against steel of the adhesive measured according to ASTM D 3330 M-99 is less than 4.0 N/25 mm.

### TECHNICAL FIELD

The present invention relates to a draping product for surgical interventions, which on its underside is coated with adhesive along at least one of its edges, said coating extending fully or partly along the edge.

### BACKGROUND OF THE INVENTION

Draping products with adhesive edge are often applied around a surgical site in order to ensure a barrier between the operative area and the part of the body of the patient that lies outside of the operative area. This barrier shall, on one hand, prevent bacteria and the like from the body of the patient to contaminate the operative area and, on the other hand, prevent blood, bacteria and the like from the operative area from contact with the body of the patient in the parts lying outside the operative area or from contaminating operating table or other operating equipment. Thus, the edge of the draping product extending adjacent to the operative area must adhere tightly against the skin and the strength of the adhesive bond must be great enough for the draping product to safely remain bonded for the loads it is normally subjected to during an operation.

Draping products can consist of individual surgical drapes provided with operative openings for a special surgical operation or sets of draping products, which can be used for different types of surgical operations, for example is a set of two surgical drapes and two surgical towels which can be draped around a operative area of a patient, on sale from Mölnlycke Health Care AB, Göteborg, Sweden, under the trade mark Klinidrape®. The adhesive edge of the surgical drapes is long and it is difficult to apply such drapes without the adhesive edge being stuck to metal parts of the operation table or being folded and adhered to itself. At certain occasions, the adhesive edge is adhered to metal racks and the like in order to fit the draping to a certain surgical operation. The draping can then need to be adjusted so that an optimal placing of the operative area shall be obtained. Doctors and nurses often complain that the adherence force of the adhesive used today in draping products, is to large against surfaces to which the adhesive edge of the draping product is not intended to be affixed. A too large adherence against metal parts, for example, can lead to that the adhesive comes off the draping product or that the draping products breaks at removal and have to be discarded.

The object of the present invention is to solve these problems and provide a draping product with adhesive edge, which safely adheres against skin but has such a small adherence force against other surfaces in an operation theatre so that the adhesive edge easily can be loosened from such surfaces.

### SUMMARY OF THE INVENTION

According to the invention, this object is achieved by a draping product, which on its underside is coated with adhesive along at least one edge thereof, said coating extending fully or partly along the edge, characterised in that the adherence force against skin of the adhesive is greater than 0.5 N/25 mm, preferably greater than 1.0 N/25 mm and more preferably greater than 1.2 N/25 mm, and that the adherence force against steel of the adhesive measured according to ASTM D 3330 M-99 is less than 4.0 N/25 mm, preferably less than 3.5 N/25 mm, more preferably less than 3.0 N/25 mm and most preferably less than 2.5 N/25 mm.

Furthermore, the invention relates to a draping product, which on its underside is coated with adhesive along at least one edge thereof, said coating extending fully or partly along the edge, characterised in that the adherence force against skin of the adhesive is greater than 0.5 N/25 mm, preferably greater than 1.0 N/25 mm and more preferably greater than 1.2 N/25 mm, and that the adherence force of the adhesive against itself measured according to ASTM D 3330 M-99 is less than 10 N/25 mm, preferably less than 8 N/25 mm, more preferably less than 6 N/25 mm and most preferably less than 5 N/25 mm.

In the preferred embodiments, the damage to Stratum Corneum of the part of the skin covered by the adhesive is after removal of a draping product attached to the skin, less than 30%, preferably less than 20% and more preferably less than 10%, measured with SCT (Spectroscopic Colour Test). At removal of the draping products with adhesive edge used today, parts of Stratum Corneum, i.e. the upper layer of the skin, stick to the adhesive and come loose from the skin when the draping product is removed. This may lead to damage of the skin, especially for patients with a sensitive skin, such as children less than three years of age and persons over seventy years of age. Such damage risks are avoided by the preferred embodiments of the invention. At a second application of the draping product, the adherence force is preferably reduced by less than 40%, more preferably by less than 30% and most preferably by less than 20%. The rests of skin affixed to the adhesives used today in adhesive edges, after removal of a draping product being applied once have the consequence that the draping product can not be securely affixed to the skin a second time. However, the draping product often needs to be loosened from its first application and reapplied on a new place on the skin, for example if the method of surgical operation is changed during an on-going operation or if the placing of the operative area need to be adjusted before the surgical operation takes place. There is then a great risk that parts of the edge of a draping product of today will be so badly affixed to the skin than the above mentioned barrier function is at risk. This risk is eliminated by the present invention.

The adhesive coating has suitably a width perpendicular to the edge of the draping product less than 150 mm.

The adhesive can consist of a pressure sensitive adhesive (PSA), for example a silicone elastomer, a hydrogel or a soft tacky hot melt adhesive.

In a variant of the preferred embodiment the adhesive is affixed to the underside of a strip of carrier material, the upper side of which being affixed to the underside of the draping product.

In an alternative embodiment, a separate strip is applied on the skin of a patient to provide a landing zone for at least a part of an edge of a draping product, the strip being characterised in that is coated on its underside with an adhesive having an adherence force against skin greater than 0.5 N/25 mm, preferably greater than 1.0 N/25 mm and more preferably greater than 1.2 N/25 mm, and that the adherence force against steel of the adhesive measured according to ASTM D 3330 M-99 is less than 4.0 N/25 mm, preferably less than 3.5 N/25 mm, more preferably less than 3.0 N/25 mm and most preferably less than 2.5 N/25 mm.

In a further alternative embodiment a separate strip is applied on the skin of a patient to provide a landing zone for at least a part of an edge of a draping product, the strip being characterised in that is coated on its underside with an adhesive having an adherence force against skin greater than 0.5 N/25 mm, preferably greater than 1.0 N/25 mm and more preferably greater than 1.2 N/25 mm, and that the adherence force of the adhesive against itself measured according to ASTM D 3330 M-99 is less than 10 N/25 mm, preferably less than 8 N/25 mm, more preferably less than 6 N/25 mm and most preferably less than N/25 mm.

On its upper side the strip can be totally smooth to provide a good foundation for the adhesive edge of a draping product. Alternatively, the strip can be provided with affixing means for affixing the strip to a draping product lacking an adhesive edge. Advantageously, the damage to Stratum Corneum of the part of the skin covered by the adhesive is, after removal of a draping product attached to the skin, less than 30%, preferably less than 20% and more preferably less than 10%, measured with SCT (Spectroscopic Colour Test). Furthermore, the strip has a width of 25-200 mm.

### BRIEF DESCRIPTION OF THE DRAWINGS

The invention will now be described with reference to the enclosed figures, of which;

FIG. 1 schematically shows a planar view from above of a draping system for four draping products according to a preferred embodiment of the invention placed around an operation opening,

FIG. 2 shows a cross section along line II-II in FIG. 1,

FIG. 3 schematically shows measuring of adherence force against skin,

FIG. 4 schematically shows draping of draping products around an operation opening using a strip according to a second embodiment of the invention, and

FIG. 5 shows a cross section along line V-V in FIG. 4.

### DESCRIPTION OF EMBODIMENTS

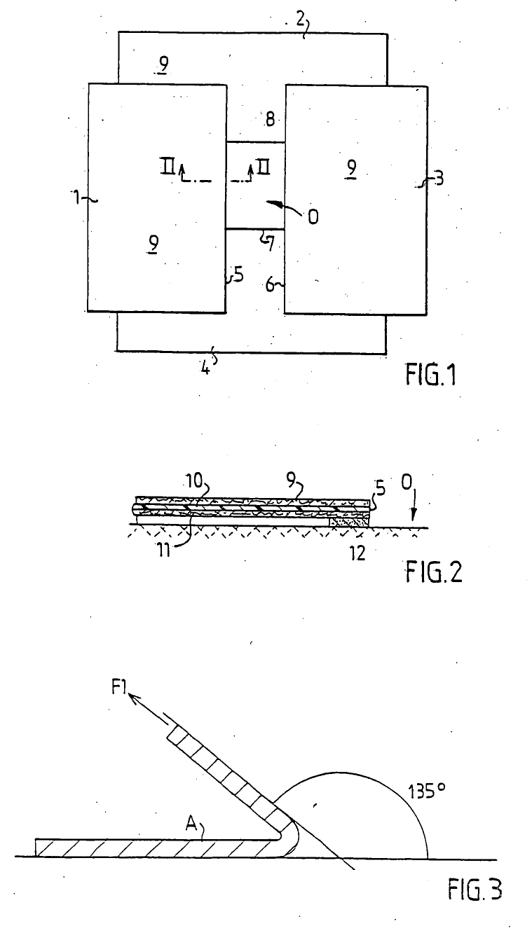

In FIG. 1 is schematically shown a draping system comprising four draping products 1-4, two surgical drapes 2 and 4 and two surgical towels 1 and 3, applied around an operative area O on a patient not shown in the figure. Draping products 1 and 3 delimit two opposite, parallel edges 5,6 of the operative area and the draping products 2 and 4 delimit two opposite, parallel edges 7,8 being perpendicular to the edges 5,6. In order to prevent liquid from the operative area from flowing under the edges 5-8 or bacteria from the area outside the operative area from penetrating into the operative area, the edges 5-8 are adhesively affixed to the skin of the patient.

The draping products 1-4 may advantageously be surgical drapes and surgical towels denoted Klinidrape® from Mölnlycke Health Care AB, Sweden, consisting of a laminate of three layers, a liquid absorbing top layer 9 of nonwoven, a liquid-tight middle layer 10 of polyethylene and a lower absorbent layer 11 of cellulose wadding or alternatively nonwoven. The top layer functions to absorb blood and other liquids emitted from the operative area and to prevent contamination of the patient from the operating staff and contamination of the operating staff and the operating theatre. The plastic film provides a barrier against liquid carried bacteria transport between the patient and the operative area and the layer of cellulose wadding enhances the comfort of the patient by absorbing sweat and preventing direct contact of the skin of the patient and the plastic film. The draping products 1-4 have also an adhesive coating 12 along their edges 5-8.

The main function of the adhesive coating 12 is to connect the draping product tightly to the skin of the patient so that liquid carried bacteria transport between the patient and the operative area is prevented and to securely fasten the draping product to the patient so that the product remains attached during all loads acting on the product during an operation. In this respect, it is pointed out that the draping products extending in the longitudinal direction of the patient normally will hang down from the operating table and thereby are the most loaded products most of the time. Furthermore, the weight of the liquid that is absorbed or in other ways is taken up by the draping product, e.g. by pouches formed in or placed on the draping product, will load the fastening area of the draping product. The adhesive coating is dimensioned to safely manage the maximal load normally acting on the draping product.

In certain occasions it can be difficult to apply draping products without the adhesive edge being stuck to metal parts of the operation table or being folded and adhered to itself. One reason for this is that the adhesive edge of the draping product is long. Another reason is that the operative area can be difficult to reach. In many cases the adhesive edge is adhered to steel racks, etc in order to provide a good setting for the surgical operation. For the application of the draping product to be easy and smooth it must be easy to loosen the adhesive edge from surfaces to which it is not intended to be attached. For this reason an adhesive according to the invention has an adherence force against surfaces normally existing in a theatre for surgical operations, such as the components of an operating table and anaesthesia arches and other steel surfaces near the operating table, that is less 4.0 N/25 mm, preferably less than 3.5 N/25 mm, and most preferably less than 2.5 N/25 mm measured in accordance with ASTM D 3330 M-99.

Furthermore, the adherence force of the adhesive against itself shall be less than 10 N/25 mm, preferably less than 8 N/25 mm, more preferably less than 5 N/25 mm. The adherence force of the adhesive against itself is also measured according to ASTM D 330 M-99, whereby a first adhesive edge is affixed to a steel plate with the adhesive facing upwards. Thereafter a second adhesive edge is applied on the first adhesive edge and is pressed against the first edge with a force with the aid of a roller having a weight of 2 kg. Then the pull force 180° is measured according to said standard.

Moreover, it can be difficult, at a first try, to get the edge, which shall be affixed to the skin of the patient, in the right location. It is therefore desirable that the adhesive coating 12 can be fastened several times against the skin without having its adherence force heavily reduced.

The adhesive in the coating shall furthermore be skin friendly and allow removal of the draping product without damage to the skin around the operative area. This requirement is a great problem with the pressure sensitive adhesives now used as adhesive coatings for draping products. Such adhesives fasten often so hard to the skin so that parts of Stratum Corneum, i.e. the upper layer of the skin, stick to the adhesive and come loose from the skin when the fastening area of the draping product is loosened. This may lead to irritation and damage of the skin, especially for patients with a sensitive skin, such as persons over seventy years of age, children less than three years of age and patients having certain illnesses, such as psoriasis, or being subjected to certain treatments, such as treatment with cortisone. For such patients the draping products must sometimes be applied without use of the adhesive edge normally provided on the product, the draping products being attached in a different manner, e.g. by attaching the draping product with the aid of several pieces of fastening tape.

The adhesive attachment of the draping product is during use of the product almost solely subjected to shear forces. Consequently, the strength of the attachment can be increased by increasing the area of adhesive coating, i.e. by increasing the width of the adhesive coating along the edge of the draping product. It has, however, been shown that the width of the adhesive coating, i.e. its extension in a direction perpendicular to the edge, should not be larger than 200 mm, preferably not larger than 150 mm. If the adhesive coating has a larger width the application of the adhesive edge of the draping product is rendered difficult to a too large extent, which can cause formation of folds that can form channels for bacteria transport and thereby cause breaks in the barrier that should be provided by the adhesive edge.

Since the properties of skin varies from person to person, the adherence force against skin of the adhesive coating will vary for different patients. The values of adherence force stated below shall be measured by a method that is schematically illustrated in FIG. 3. Strips A of a carrier material coated with an adhesive, the adherence force of which is to be measured, and having a width of 25 mm are placed on the back of at least ten healthy persons of varying ages and sex and is maintained fastened to the skin during two minutes. Thereafter, the strips A are pulled with a rate of 25 mm/sec and the pulling force F1 is measured. The pulling angle, i.e. the obtuse angle formed between the skin surface and the pulled off portion of the strip A, shall be 135°. The adherence force against skin of the measured adhesive consists of the mean value of the force F1. Adhesives that can be used in a draping product according to the invention shall have an adherence force of at least 0.5 N/25 mm.

Measurements of the adherence force against skin, against steel and against itself was performed in the above described manner for adhesive coated strips from the commercially accessible products Klinidrape® Universal Set Basic, art. No. 698740,from Mölnlycke Health Care AB, Göteborg, Sweden, Allegiance Convertors, REF 2915CE from McGaw Park, Ill., USA and 3M Steri-Drape, 9000 from 3M, St. Paul, Minn., USA and for a strip of Klinidrape®-material, to which a strip of polyurethane was laminated and coated with the elastomer Silgel 612 from Wacker Chemie GmbH, Germany. The result of these measurements are shown in table 1.

TABLE 1

| | | Adhesive | Adhesive | |
|---|---|---|---|---|
| | | Klinidrape, | Allegiance, | Adhesive |
| | Silgel 612 | U-set | U-set | 3M, U-set |
| Fₘₑₐₙᵥₐₗᵤₑ against | 1.14 | 0.50 | 0.52 | 0.60 |
| skin, N/25 mm | | | | |
| Fₘₑₐₙᵥₐₗᵤₑ against | 1.03 | >15 | >15 | >15 |
| itself, N/25 mm | | | | |
| Fₘₑₐₙᵥₐₗᵤₑ against | 2.43 | 5.57 | 6.88 | 7.70 |
| steel plate, | | | | |
| N/25 mm | | | | |

It is evident from the results that only Silgel 612 of the tested adhesives fulfils the demands that can be made for suitable adhesives for draping products with adhesive edge. The measurements for the values >15 given in the table could not be finalised since the strips broke or the adhesive delaminated from the strip before the adherence of the adhesive against itself loosened.

Moreover, the damage to Stratum Corneum of the part of the skin covered by the adhesive shall after removal of a draping product attached to the skin be less than 30%, preferably less than 20% and more preferably less than 10%, measured with SCT (Spectroscopic Colour Test). The SCT-measuring shall be made in the way described in detail in P. J. Dykes, R. Heggie, S. A Hill, “effects of adhesive dressings on the stratum corneum of the skin”, Journal of wound care, February, Vol 10, No. 2, 2001, which article is referred to for further details. The SCT-measuring shall be performed on at least ten person of varying sex and having healthy skin and be performed in the following way. Firstly, the skin in the centre of the test zone is coloured by application of a 12 mm aluminium Finn chamber containing a 11 mm filter paper disc wetted with a 0.03 ml 1% aqueous methylene blue. The Finn chamber shall be applied to the skin surface for 60 minutes. This is sufficient to produce an even colouring of the superficial layers of the Stratum Corneum. Then, the test strips are applied to the coloured zones of the skin of the test persons and are applied thereon for 72 hours. After removal of the test strips after 72 hours, the stratum corneum shall be removed by “Skin surface biopsy procedure”, which is described in R. Marks, R. P. R. Dawber, “Skin surface biopsy; an improved technique for examination of the horny layer”, Br. J Dermatol 1971:84:117-123, to which is referred for further details. The biopsies shall thereafter be cut into smaller pieces and be placed in glass tubes containing 2 ml dimethyl sulphoxide (DMSO). The glass tubes shall be shaken every 10-15 minutes over a period of two hours to ensure the dye extraction to be complete. The dimethyl sulphoxide extract shall then be centrifuged at 100 g for 10 minutes to remove all fragments of stratum corneum. One millilitre of dimethyl sulphoxide shall then be transferred to a plastic cuvette to measure the optical density. The optical density shall be measured with a spectrophotometer. Initially, a blank cuvette containing dimethyl sulphoxide shall be scanned from 550-800 nm. Thereafter, an extracted skin surface biopsy from a coloured zone of the skin be scanned to determine the maximal absorbance. All subsequent measurements shall be performed at the wave length for maximal absorbance. The results are expressed as optical density units and are presented as a percentage of damage of Stratum Corneum relative to a reference sample of adjacent undamaged Stratum Corneum.

In table 1 below, the damage of Stratum Corneum caused by removal of adhesive from skin and measured by the above described spectroscopic colouring method (SCT) is shown for several different known products provided with adhesive; Allevyn from Smith & Nephew, Hull, Great Britain, Tielle hydropolymer dressing from Johnson & Johnson, Gargrave, Great Britain, Duoderm Extra Thin from ConvaTec Ltd, Deeside, Great Britain, Mepilex Border from Mölnlycke Health Care AB, Göteborg, Sweden and Biatain from Coloplast, Humlebaeck, Denmark.

TABLE 1

| | Damage of Stratum |
|---|---|
| | corneum (%) |
| Allevyn adhesive | 96.4 |
| Tielle hydropolymer adhesive | 90.9 |
| Duoderm | 81.8 |
| Mepilex Border adhesive | −1.8 |
| Biatain adhesive | 87.3 |

From table 1 it is evident that only the adhesive on Mepilex Border fulfils the above mentioned requirements, said adhesive consisting of a silicone adhesive, elastomer Silgel 612 from Wacker Chemie GmbH, Germany. The negative value of skin damage is probably an effect of spreading of measuring data but can also relate to that the adhesive acts as a protection from the natural abrading of skin cells compared to the reference sample only being covered by gauze fabric during the measuring period.

It has been shown that strips with an adhesive having an adherence force of 0.5 N/25 mm and giving a damage to Stratum Corneum of the part of the skin covered by the adhesive being less than 10% after removal of a draping product attached to the skin measured with SCT (Spectroscopic Colour Test) can be applied also to patients having sensitive skin and be removed without damage to or irritation of the skin.

Examples of adhesives that can be used for draping products according to the present invention are silicone elastomers, for example a silicone elastomer for sale under the name Silgel 612 manufactured by Wacker Chemie GmbH, Germany, or soft, tacky hot melt adhesives. Silicone elastomers have further the advantage of being hydrophobic, which ensures that the edge of the draping product can be sealingly attached to the skin. It is also conceivable to use hydrogels of a type that tightly connects to the three-dimensional structure of the skin and in this manner prevents passage of liquid through the adhesive coating and not or only to small extent admit diffusion of liquid out of the hydrogel. However, the use of hydrophobic adhesives is preferred.

It has been shown that a silicone elastomer with an adherence force of 1.4 N/25 mm also fulfils the requirement that a damage to Stratum Corneum of the part of the skin covered by the adhesive is less than 10% after removal of a draping product attached to the skin measured with SCT (Spectroscopic Colour Test). Such an elastomer is thus very suitable to use in draping products.

A reason for soft adhesives to function well for affixing draping products to skin is that the softness makes it possible for the adhesive to fill all irregularities of the skin. This means that the adhesive covers a much larger part of the skin than the types of adhesives being used today for affixing draping products to the skin. Since a larger surface of the skin is used for attachment of a soft adhesive, the adherence to skin can be smaller than for a harder adhesive and still the soft adhesive can have a larger adherence force in N/25 mm than the harder adhesive. This allows the soft adhesive to be pulled off the skin without other than loose parts of Stratum Corneum sticking to the adhesive.

In order to reduce the necessary width/length unit of the adhesive coating 12 and increase the safety margin in use of the draping products 1-4, the adherence force to skin of the adhesive coating 12 is advantageously larger than 1.0 N/25 mm, preferably larger than 1.2 N/25 mm.

With the hard adhesives used today for affixing draping products, the adhesive coating of the draping products is to a large extent covered by Stratum Corneum cells after removal from the skin. This leads to that their adhesive capacitance is reduced to such an extent that the edge of the draping product can not be reapplied to the skin. The application of such a draping product requires great caution since an incorrectly applied draping product can not in a simple manner be loosened and reapplied on a correct location but should be substituted by a fresh draping product.

Measurement of adherence force against skin with reapplication is made in the following way. Test strips are applied on the back of ten persons of varying age and sex having a healthy skin. After two minutes the strips are pulled off in the same manner as described above with reference to FIG. 3 at a rate of 25 mm/sec and the pull force F1 is measured. The pull angle shall be 135°. The strip is then reapplied on the skin on an untouched spot on the back and after two minutes the measurement of the pull force is repeated, wherewith the pull force F2 is obtained. The reduction of the pull force at the second pull relative to the first shall be less than 40%, preferably less than 30% and more preferably less than 20% and the pull force F2 at the second pull shall be larger than 0.5 N/25 mm, preferably larger than 1.0 N/25 mm and more preferably larger than 1.2 N/25 mm.

Such a measurement was performed for adhesive coated strips from the commercially accessible products Klinidrape® Universal Set Basic, art. No. 698740, from Mölnlycke Health Care AB, Göteborg, Sweden, Allegiance Convertors, REF 2915CE from McGaw Park, Ill., USA and 3M Steri-Drape, 9000 from 3M, St. Paul, Minn. USA and for a strip of Klinidrape®-material, to which a strip of polyurethane was laminated and coated with the elastomer Silgel 612 from Wacker Chemie GmbH, Germany. The result of these measurements are shown in table 2 below.

TABLE 2

| | | | | 3M |
|---|---|---|---|---|
| | Silicone | Klinidrape | Allegiance | adhesive |
| | adhesive | adhesive, U-set | adhesive, U-set | U-set |
| Fₘₐₓ first | 1.71 | 0.72 | 0.80 | 0.82 |
| (N/25 mm) | | | | |
| Fₘₐₓ second | 1.44 | 0.35 | 0.37 | 0.41 |
| (N/25 mm) | | | | |
| Reduction of | 16 | 51 | 54 | 50 |
| adherence force | | | | |
| (%) | | | | |
| Fₘₑₐₙᵥₐₗᵤₑ first | 1.14 | 0.50 | 0.52 | 0.60 |
| (N/25 mm) | | | | |
| Fₘₑₐₙᵥₐₗᵤₑ | 1.00 | 0.24 | 0.24 | 0.28 |
| second | | | | |
| (N/25 mm) | | | | |
| Reduction of | 12 | 51 | 53 | 53 |
| adherence force | | | | |
| (%) | | | | |

These measurements makes it clear that Silgel 612 can function well as an adhesive for a draping product having an adhesive edge.

In the embodiment disclosed in FIGS. 1 and 2, the layer 12 of cellulose wadding is not extended along the edge of the draping product and the adhesive coating 12 is affixed to the plastic layer 10. In order to ensure that the adherence force of the adhesive against the underside of the draping product is larger than the adherence force against skin it is possible to affix the adhesive coating to a strip of material, e.g. a nonwoven or an appropriate plastic material, to which the adhesive coating surely adheres with a larger force than against skin, and in turn affix the strip to the underside of draping product material with an adhesive that adheres well to the draping product material and to the strip. The strip can also consist of a laminate of a plastic film and a nonwoven, the nonwoven layer facing the adhesive coating, the plastic layer of the strip is affixed to the draping product material in an appropriate way, for example by glue. It is, however, preferred to affix the adhesive coating 12 directly to the underside of the draping product.

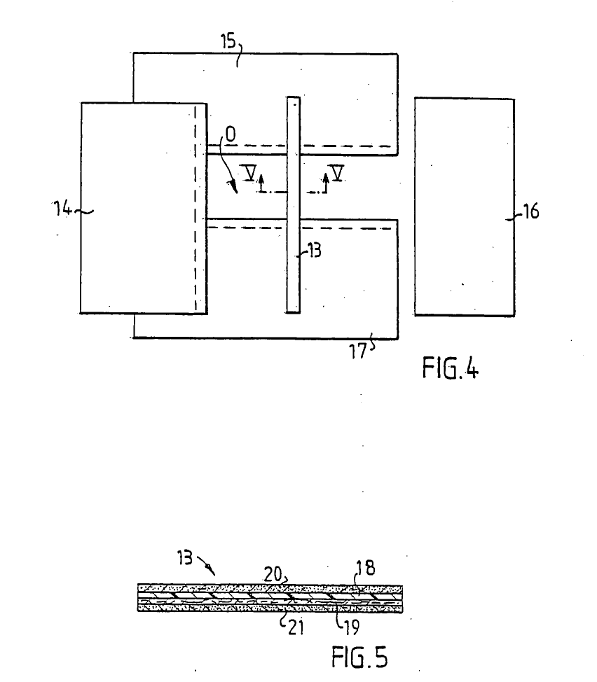

A second embodiment of the invention is disclosed in FIGS. 4 and 5, in which separate strips 13 coated with adhesive is used together with draping products 14-17 for accomplishing a similar draping of draping products around an operative area O as shown in FIG. 1. The strips 13 preferably consist of a plastic film 18, which on its upper side is coated with an adhesive 19 adhering well to the draping product material, e.g. an acrylate glue, and on its underside is coated with an adhesive 20 adhering well to skin. The adhesive 20 is an adhesive having the same properties as the adhesive 12 in the embodiment described above with reference to FIGS. 1 and 2. Consequently, the draping products 14-17 lack adhesive coating. In a draping procedure with the aid of such a combination of draping product and strip, the strip is first applied along an edge of the operative area O, whereafter the edge of the draping product is applied to the strip and thereby is affixed to the adhesive 20 of the strip. By the fact that the draping product lacks adhesive coating that can stick to the product or wrong parts of the patient's body it is very easy to handle such a draping product and apply it to already affixed strip. In spite of this arrangement being a two-step procedure, the application of the draping product is facilitated in such an extent that the time for application will not be longer than for the application of draping products having an integrated adhesive edge. In FIG. 4, the draping of the draping products 14-17 is shown in a final step, in which only the affixing of the edge of the draping product 16 to a strip 13 remains to be done.

The strip 13 should be made by a material, to which the adhesive facing the skin side, adheres with a greater force than to skin, and can in addition to a plastic layer consist of a nonwoven or a laminate of a nonwoven and a plastic layer. In order to allow a secure and easy application, the width of the strips 13 should preferably be 25-200 mm.

In an alternative embodiment of the strips 13, these lack adhesive coating on their upper side and co-operates with draping products 14-17 having adhesive edge of conventional type.

It is pointed out that all values of adherence force relate to adherence to dry skin. Moreover, all adhesive coatings of draping products or strips are provided with protective release layers, which are to be removed before application.

By the underside of the draping products or the strips is meant the side facing the body of a patient during use of the draping products or the trips.

The present invention can of course be used for other types of draping products than the products described in the embodiments, for example for surgical drapes having pre-prepared operative opening, around which an adhesive edge or adhesive edges extend. The scope of the present invention shall therefore include all known types of draping products intended to be affixed to the body of a patient.

The disclosed embodiments can of course be modified within the scope of the invention. The draping products can be manufactured of other materials than the described draping products, e.g. consist of one-layered draping products of textile or textile-like materials. The scope of invention shall therefore only be restricted by the content of the enclosed patent claims.

1. A draping product (1-4) for surgical interventions, which on its underside is coated with adhesive along at least one edge thereof (5,6, 7 and 8, respectively), said coating (12) extending fully or partly along the edge, characterised in that the adherence force against skin of the adhesive is greater than 0.5 N/25 mm, preferably greater than 1.0 N/25 mm and more preferably greater than 1.2 N/25 mm, and that the adherence force against steel of the adhesive measured according to ASTM D 3330 M-99 is less than 4.0 N/25 mm, preferably less than 3.5 N/25 mm, more preferably less than 3.0 N/25 mm and most preferably less than 2.5 N/25 mm.

2. A draping product (1-4) for surgical interventions, which on its underside is coated with adhesive along at least one edge (5,6, 7 and 8, respectively) thereof, said coating (12) extending fully or partly along the edge, characterised in that the adherence force against skin of the adhesive is greater than 0.5 N/25 mm, preferably greater than 1.0 N/25 mm and more preferably greater than 1.2 N/25 mm, and that the adherence force of the adhesive against itself measured according to ASTM D 3330 M-99 is less than 10 N/25 mm, preferably less than 8 N/25 mm, more preferably less than 6 N/25 mm and most preferably less than 5 N/25 mm.

3. A draping product according to claim 1, characterised in that the damage to Stratum Corneum of the part of the skin covered by the adhesive is, after removal of a draping product attached to the skin, less than 30%, preferably less than 20% and more preferably less than 10%, measured with SCT (Spectroscopic Colour Test).

4. A draping product according to claim 1, characterised in that the adhesive coating (12) has a width perpendicular to the edge of the draping product (1-4) less than 150 mm.

5. A draping product according to claim 1, characterised in that adhesive consists of a silicone elastomer, a hydrogel or a soft, tacky hot melt adhesive.

6. A draping product according to claim 1, characterised in that the adhesive is affixed to the underside of a strip of carrier material, the upper side of which being affixed to the underside of the draping product.

7. A draping product according to claim 1, characterised in that, at a second application of the draping product against skin, the adherence force against skin of the adhesive is reduced by less than 40%, preferably less than 30% and more preferably less than 20% .

8. A strip (13) for affixing at least a part of an edge of a draping product (14-17) to the skin of a patient, characterised in that the strip (13) on its underside is coated with an adhesive having an adherence force against skin greater than 0.5 N/25 mm, preferably greater than 1.0 N/25 mm and more preferably greater than 1.2 N/25 mm, and that the adherence force against steel of the adhesive measured according to ASTM D 3330 M-99 is less than 4.0 N/25 mm, preferably less than 3.5 N/25 mm, more preferably less than 3.0 N/25 mm and most preferably less than 2.5 N/25 mm.

9. A strip (13) for affixing at least a part of an edge of a draping product (14-17) to the skin of a patient, characterised in that the strip (13) on its underside is coated with an adhesive having an adherence force against skin greater than 0.5 N/25 mm, preferably greater than 1.0 N/25 mm and more preferably greater than 1.2 N/25 mm, and that the adherence force of the adhesive against itself measured according to ASTM D 330 M-99 is less than 10 N/25 mm, preferably less than 8 N/25 mm, more preferably less than 6 N/25 mm and most preferably less than 5 N/25 mm.

10. A strip according to claim 8, characterised in that it on its upper side is provided with affixing means (20) for affixing the strip to a draping product (14-17).

11. A strip according to claim 8, characterised in that the damage to Stratum Corneum of the part of the skin covered by the adhesive is, after removal of a draping product attached to the skin, less than 30%, preferably less than 20% and more preferably less than 10%, measured with SCT (Spectroscopic Colour Test).

12. A strip according to claim 8, characterised in that the strip (13) has a width of 25-200 mm.

13. A strip according to claim 9, characterised in that it on its upper side is provided with affixing means (20) for affixing the strip to a draping product (14-17).

14. A draping product according to claim 2, characterised in that the damage to Stratum Corneum of the part of the skin covered by the adhesive is, after removal of a draping product attached to the skin, less than 30%, preferably less than 20% and more preferably less than 10%, measured with SCT (Spectroscopic Colour Test).
